(19) United States
(12) Patent Application Publication
(10) Pub. No.: US 2017/0000886 A1
(43) Pub. Date: Jan. 5, 2017

(54) MESENCHYMAL STEM CELLS FOR TARGETED CANCER THERAPY

(71) Applicant: THE REGENTS OF THE UNIVERSITY OF CALIFORNIA, Oakland, CA (US)

(72) Inventors: Astra Chang, Davis, CA (US); Jan Nolta, Davis, CA (US)

(21) Appl. No.: 15/189,853

(22) Filed: Jun. 22, 2016

Related U.S. Application Data
(63) Continuation of application No. PCT/US2014/071990, filed on Dec. 22, 2014.
(60) Provisional application No. 61/920,435, filed on Dec. 23, 2013.

(51) Int. Cl. A61K 39/395; C12N 15/86; A61K 38/20; A61K 48/00; C07K 16/28; C07K 14/55; C12N 5/0775; C12N 7/00

(52) U.S. Cl. CPC A61K 39/39558; C12N 5/0668; C12N 15/86; C12N 7/00; A61K 48/0066; C07K 16/2884; C07K 14/55; A61K 38/2013; C12N 2740/15043; C07K 2317/52; C07K 2317/622; C07K 2317/21; C07K 2319/02; C07K 2319/30; A61K 2039/505

(57) ABSTRACT

An isolated polynucleotide comprising the element of: a promoter element that drives expression of the C—C motif ligand 5 (“a CCL5 promoter”) operatively linked to a polynucleotide encoding a fusion polypeptide comprising the Fc and hinge regions of a human IgG CD44 variant 6 (CD44v6) polypeptide is described.

### CROSS-REFERENCE TO RELATED APPLICATIONS

This application is a continuation under 35 U.S.C. §120 of International Application No. PCT/US2014/071990, filed Dec. 22, 2014, claims priority under 35 U.S.C. §119(e) to U.S. Provisional Patent Application No. 61/920,435, filed Dec. 23, 2013, the content of which is incorporated herein by reference in its entirety.

### BACKGROUND

Although significant advances have been made in the war on cancer, the disease is remains a major global health problem. One in 4 deaths in the United States is due to cancer. (Siegel, R. et al. (2013) CA Cancer J. Clin. 63(1):11-40). The American Cancer Society projected that in 2013; about 580,350 Americans were expected to die of cancer, almost 1,600 people per day. Cancer is the second most common cause of death in the U.S., exceeded only by heart disease, accounting for nearly 1 of every 4 deaths. Thus, a need exists for effective treatments for this still fatal disease. This disclosure satisfies this need and provides related advantages as well.

### SUMMARY OF THE DISCLOSURE

Mesenchymal stem/stromal (MSC) cells have been demonstrated to partake in cellular communication with tumor cells of various different types. This back and forth communication is believed to enable cancer stem cells (CSC) to increase their mobility and enhance metastatic potential. The cellular dialogue includes the summoning of MSC by cancer cells via a poorly understood signaling mechanism related to hypoxic conditions and inflammatory conditions. As MSC approach the tumor bed they are stimulated to upregulate secretion of CCL5/RANTES (CCL5), which the cancer cells in part utilize to guide to escape their environment and metastasize. In order to disrupt the metastatic process of cancer stem cells, a Trojan horse approach was utilized in a design to selectively target CSC. By engineering MSC to secrete an antibody-like molecule that specifically targets the metastatic cancer variant of CD44 termed CD44v6, under the controlled expression of the CCL5 promoter, a “Mesenkiller” cell is generated. Herein, it is shown that cancer cells are effectively able to attract the engineered Mesenkiller cells, stimulate the CCL5 promoter and the expression and secretion of engineered antibody-like molecules that are specific to CD44v6, and would be effective in initiating an innate immune system through the opsonization of the target and stimulation of innate immune cells and the complement cascade. This novel approach in harnessing the intrinsic ability of MSC to home in to CSCs and regulating the secretion of an antibody-like molecule, under a promoter that is stimulated by cancer cells, has resulted in a potential biotherapeutic that may ultimately lead to a means of addressing, as well as eliminating early cancer cell metastasis.

Thus, in one aspect, this disclosure provides an isolated polynucleotide comprising, or alternatively consisting essentially of, or yet further consisting of, a promoter element that drives expression of the C—C motif ligand 5 (“a CCL5 promoter”) operatively linked to a polynucleotide encoding a fusion polypeptide, the fusion polypeptide comprising the Fc (constant region) of a human antibody selected from the group: IgG1, IgG2, IgG3, IgG4, IgA1, IgA2 or IgM polypeptide and a ScFv region of an anti-CD44 variant, e.g., CD44 variant 6 (CD44v6) polypeptide. In one aspect, the isolated polynucleotide further comprises, or alternatively consists essentially of, or yet further consists of, and an antibody hinge region, e.g., a human antibody hinge region. In a further aspect, the isolated polynucleotide further comprises, or alternatively consists essentially of, or yet further consists of, a polynucleotide encoding the signal sequence of a secreted protein. Vectors and/or host cells can comprise the polynucleotides as described herein. In one aspect, the vector is a lentiviral vector. As is apparent to those skilled in the art, other ScFv regions of antibodies directed to cell surface markers present on tumor cells can be substituted for the ScFv region of the anti-CD44 marker as disclosed herein, e.g, the ScFv region of the Herceptin receptor (HER-2). Thus, this disclosure also provides these alternative polynucleotides, vectors and host cell containing same. Compositions are further provided that comprise, or alternatively consists essentially of, or yet further consist of, one or more of the polynucleotides, vectors, and/or host cells and a carrier, for example a pharmaceutically acceptable carrier.

The compositions are useful in methods for delivering the encoded fusion polypeptide to a tumor cell expressing a CD44 marker, in one aspect a CD44 variant, e.g., CD44v6, the method comprising, or alternatively consisting essentially of, or yet further consisting of, contacting the tumor cell with an effective amount of the encoded polypeptide. Alternatively, the polynucleotide can be inserted into a host cell such as a mesenchymal stem cell and the cell is administered to a subject in need of therapy. Thus, depending on the use, the contacting can be performed, in vitro, ex vivo or in vivo. When the cells contacting is in vivo, the cells can be autologous or allogeneic to the subject receiving them.

The compositions describe above are useful in methods for one or more of inducing an immune response; for inhibiting the growth of a tumor in a subject in need thereof and for treating a cancer expressing the CD44 marker, in one aspect expressing a CD44 variant, e.g., CD44v6. The methods comprise, or alternatively consist essentially of, or yet further consist of, administration of an effective amount of the isolated host cell containing the polynucleotide expressing the fusion polypeptide as described above, in particular when the host cell is a mesenchymal stem cell or a population of mesenchymal stem cells. Subjects treated by the methods include a mammal, e.g., a simian, a bovine, a canine, a feline, an equine or a human. In these methods, the isolated host cell is autologous or allogeneic to the subject.

### BRIEF DESCRIPTION OF THE FIGURES

FIG. 1 shows cancer stem cell markers of over forty common cancer types. CD44 appears to be one of the most common cancer stem cell markers. CD34+, while a close second is limited to blood cancers.

FIG. 2 shows under agar assay gel set up showing a pattern of punched wells. The labels of each well are an example of how an experiment may be set up, and is actually the set up used for the time-lapse images shown in FIG. 5.

FIG. 3 shows schematic of the lentiviral vector design used to transduce MSC to the Mesenkiller phenotype.

FIGS. 4A-4F shows induction of CCL5 (RANTES) expression and secretion from MSC with coculture with MDA-MB-231 cancer cells. A) and B) Fluorescence microscopy of MDA-MB-231 cells loaded with Cell Trace CFSE (Invitrogen) prior to co-culture (FITC and overlay with phase, respectively). C) Unlabeled MSC D) co-culture. 100× E) Increases in expression of CCL5 message after separation by qRT-PCR following coculture. F) Increases in secreted CCL5 in the supernatant of the coculture as measured by cytometric bead array (BD Biosciences).

FIG. 5A shows time-lapse microscopy demonstrating that MDA-MB-231 cancer cells attract MSC. GFP+MSC can be seen migrating towards a cluster of cancer cells (off-screen below). Rather than migrate up to meet these cells, the cancer cells are attracted towards MSC grown in conditioned media (in FIG. 5B). 100×

FIG. 5B shows time-lapse microscopy demonstrating that MDA-MB-231 will be chemoattracted toward MSC cultured in conditioned media. 100×

FIG. 5C shows time-lapse microscopy demonstrating that MSC are more strongly chemoattracted toward IL-6 (known to be secreted by cancer cells) over OPN (also highly expressed in malignant tumors. Underagar assay was set up with MSC in the middle, OPN to the left, and IL-6 to the right.

FIGS. 6A-6D shows fluorescence microscopy showing expression of the CFP (fused with the antibody-like molecule) and the mCherry reporter gene in MSC transduced with the vector having the EF1α promoter. A) phase, B) mCherry, C) CFP, D) overlay; 200×.

FIGS. 7A-7F shows fluorescence microscopy imaging of MDA-MB-231 cells labeled with secreted engineered fusion protein/antibody-like molecule. (A, D=phase; B, E=CFP; C, F=overlay.) Arrow highlights pseudopodia-like projection that is covered with CFP labeled fusion molecule.

FIG. 8 shows fluorescence microscopy demonstrating cancer stimulated expression of mCherry, placed into the vector as a reporter for the CCL5-promoter-regulated expression of the antibody from engineered MSC. Top panels are at day 0 of the co-culture and bottom panels are at days 2 and 3. (Green=GFP+MSC, Red=mCherry, unlabeled cells are MDA-MB-231 cancer cells.)

### BRIEF DESCRIPTION OF THE SEQUENCE LISTING

SEQ. ID NO: 1 is an exemplary vector polynucleotide encoding the fusion polypeptide of this disclosure. The psi polynucleotide is from nt 685-822. The termini of additional components are referenced herein.

SEQ. ID NO: 2 is an exemplary sequence for mRNA encoding human CD44v6. An additional example is from nt 3648 to 4049 of SEQ ID NO. 1.

SEQ. ID NO: 3 is an exemplary sequence for mRNA encoding human Fc IgG1heavy chain constant region (mRNA), also available at GenBank: JN222933.1.

SEQ. ID NO: 4 is an exemplary polynucleotide encoding human IgG1 Fc fragment (mRNA), also available at GenBank: AF150959.1.

SEQ ID NO: 5 is an exemplary sequence for a polynucleotide encoding wild-type IgG2.

SEQ ID NO: 6 is an exemplary sequence for a polynucleotide encoding IgG3.

SEQ ID NO: 7 is an exemplary sequence for a polynucleotide encoding IgG4.

SEQ ID NO: 8 is an exemplary sequence for a polynucleotide encoding IgA1.

SEQ ID NO: 9 is an exemplary sequence for a polynucleotide encoding IgA2.

SEQ ID NO: 10 is an exemplary sequence for a polynucleotide encoding IgGM.

SEQ ID NO: 11 is a forward primer sequence.

SEQ ID NO: 12 is a reverse primer sequence.

SEQ ID NO: 13 is an exemplary WPRE polynucleotide.

### DETAILED DESCRIPTION OF THE INVENTION

Throughout this disclosure, various publications, patents and published patent specifications are referenced herein. The disclosures of these publications, patents and published patent specifications are hereby incorporated by reference in their entirety into the present disclosure.

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; Animal Cell Culture (R. I. Freshney, ed. (1987)); Zigova, Sanberg and Sanchez-Ramos, eds. (2002) Neural Stem Cells.

All numerical designations, e.g., pH, temperature, time, concentration, and molecular weight, including ranges, are approximations which are varied (+) or (−) by increments of 0.1 or 1 where appropriate. It is to be understood, although not always explicitly stated that all numerical designations are preceded by the term “about”. The term “about” also includes the exact value “X” in addition to minor increments of “X” such as “X+0.1 or 1” or “X−0.1 or 1,” where appropriate. It also is to be understood, although not always explicitly stated, that the reagents described herein are merely exemplary and that equivalents of such are known in the art.

### DEFINITIONS

As used in the specification and claims, the singular form “a”, “an” and “the” include plural references unless the context clearly dictates otherwise. For example, the term “a cell” includes a plurality of cells, including mixtures thereof.

The terms “administer” or “administration” or “administering” shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments, which are not naturally occurring as fragments and would not be found in the natural state. The term “isolated” is also used herein to refer to cells or polypeptides which are isolated from other cellular proteins or tissues. Isolated polypeptides are meant to encompass both purified and recombinant polypeptides.

The term “isolated” as used with respect to cells, in particular stem cells, such as mesenchymal stem cells, refers to cells separated from other cells or tissue that are present in the natural tissue in the body.

A “subject,” “individual” or “patient” is used interchangeably herein and refers to a vertebrate, for example a primate, a mammal or preferably a human. Mammals include, but are not limited to equines, canines, bovines, ovines, murines, rats, simians, humans, farm animals, sport animals and pets.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “CD44” intends a cell-surface glycoprotein involved in cell-cell interactions, cell adhesion and migration. In humans, the CD44 antigen is involved is encoded by the CD44 gene on Chromosome 11. The polynucleotide sequence, variants and homologs thereof are known in the art and reported at the web page genecards.org/cgi-bin/carddispl.pl?gene=CD44, last accessed on Dec. 15, 2014. CD44v6 is one example of a CD44varian isoform which contains the variant in exon 6. In one aspect, biological equivalents of CD44v6 maintain the wild-type sequence of in exon 6 of the CD44v6. In another aspect, the CD44v6 exon is altered.

“Amplify” “amplifying” or “amplification” of a polynucleotide sequence includes methods such as traditional cloning methodologies, PCR, ligation amplification (or ligase chain reaction, LCR) or other amplification methods. These methods are known and practiced in the art. See, e.g., U.S. Pat. Nos. 4,683,195 and 4,683,202 and Munemitsu, S. et al. (1990) Mol. Cell Biol. 10(11):5977-5982 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “genotype” refers to the specific allelic composition of an entire cell, a certain gene or a specific polynucleotide region of a genome, whereas the term “phenotype’ refers to the detectable outward manifestations of a specific genotype.

As used herein, the term “gene” or “recombinant gene” refers to a nucleic acid molecule comprising an open reading frame and including at least one exon and (optionally) an intron sequence. A gene may also refer to a polymorphic or a mutant form or allele of a gene.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on May 21, 2008. Biologically equivalent polynucleotides are those having the specified percent homology and in one aspect, encode a polypeptide having the same or similar biological activity as the reference polynucleotide.

The term “an equivalent nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

An equivalent or biological equivalent nucleic acid, polynucleotide or oligonucleotide or peptide is one having at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, oligonucleotide or peptide. In a further aspect, the equivalent or biological equivalent nucleic acid, polynucleotide or oligonucleotide encodes a polypeptide having the identical or similar biological activity of the reference polynucleotide. In one aspect, the equivalent peptide is one having at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference peptide and the identical or similar biological activity as the reference peptide.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, or nucleic acid-nucleic acid in nature.

“Hybridization” refers to a reaction in which one or more polynucleotides react to form a hybridization complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The term “mismatches” refers to hybridized nucleic acid duplexes which are not 100% homologous. The lack of total homology may be due to deletions, insertions, inversions, substitutions or frameshift mutations.

As used herein, the term “oligonucleotide” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

As used herein, the term “fustion polypeptide” intends a non-naturally occurring polypeptide or protein that is a combination of two or more amino acids that are not found in nature. In one aspect, the fusion polypeptide is the expression produce of a non-naturally occurring polynucleotide, e.g., cDNA.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

As used herein, the term “carrier” encompasses any of the standard carriers, such as a phosphate buffered saline solution, buffers, water, and emulsions, such as an oil/water or water/oil emulsion, and various types of wetting agents. The compositions also can include stabilizers and preservatives. For examples of carriers, stabilizers and adjuvants, see Sambrook and Russell (2001), supra. Those skilled in the art will know many other suitable carriers for binding polynucleotides, or will be able to ascertain the same by use of routine experimentation. In one aspect of the invention, the carrier is a buffered solution such as, but not limited to, a PCR buffer solution.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, H. et al. (1999) Nat. Med. 5(7):823-827.

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form, which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono, D. (2002) Lentiviral vectors, New York: Spring-Verlag Berlin Heidelberg.

The term “express” refers to the production of a gene product. In some embodiments, the gene product is a polypeptide or protein.

A cell that “stably expresses” an exogenous polypeptide is one that continues to express a polypeptide encoded by an exogenous gene introduced into the cell either after replication if the cell is dividing or for longer than a day, up to about a week, up to about two weeks, up to three weeks, up to four weeks, for several weeks, up to a month, up to two months, up to three months, for several months, up to a year or more.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, H. et al. (1999) Nat. Med. 5(7):823-827.

As used herein, the term “signal sequence” intends a polypeptide that is found at the amino terminus of a nascent protein, and functions by prompting cellular transport to the secretory pathway. Polypeptides and polynucleotides (DNA and RNA) encoding such are know in the art and can be found at “Signal Peptide Website: An Information Platform for Signal Sequences and Signal Peptides”, at the web address: signalpeptide.com, last accessed on Dec. 12, 2013.

In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, retroviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application Publication No. WO 95/27071. Ads do not require integration into the host cell genome. Recombinant Ad derived vectors, particularly those that reduce the potential for recombination and generation of wild-type virus, have also been constructed. See, International PCT Application Publication Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski, J. S. et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells.

The term “promoter” refers to a region of DNA that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible and as used herein, the promoter can be constitutive or inducible. A constitutive promoter refers to one that is always active and/or constantly directs transcription of a gene above a basal level of transcription. An inducible promoter is one which is capable of being induced by a molecule or a factor added to the cell or expressed in the cell. An inducible promoter may still produce a basal level of transcription in the absence of induction, but induction typically leads to significantly more production of the protein. Promoters can also be tissue specific. A tissue specific promoter allows for the production of a protein in a certain population of cells that have the appropriate transcriptional factors to activate the promoter. Promoters useful in this disclosure can be constitutive or inducible. Some examples of promoters include SV40 early promoter, mouse mammary tumor virus LTR promoter, adenovirus major late promoter, herpes simplex virus promoter, and the CMV promoter.

An “enhancer” is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. In one aspect, the enhancer is a Woodchuck post-regulatory element (“WPRE”) (see, e.g., Zufferey, R. et al. (1999) J. Virol. 73(4):2886-2992) and nt 6807-7398 of SEQ ID NO. 1 and equivalents thereof having enhancer function.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels are described and exemplified herein.

A “primer” is a short polynucleotide, generally with a free 3′ —OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson, M. et al. (1991) PCR: A Practical Approach, IRL Press at Oxford University Press. All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra. The primers may optionally contain detectable labels and are exemplified and described herein.

As used herein, the term “label” or “detectable label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP), yellow fluorescent protein, red fluorescent protein, and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to any one of a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

For topical use, the pharmaceutically acceptable carrier is suitable for manufacture of creams, ointments, jellies, gels, solutions, suspensions, etc. Such carriers are conventional in the art, e.g., for topical administration with polyethylene glycol (PEG). These formulations may optionally comprise additional pharmaceutically acceptable ingredients such as diluents, stabilizers, and/or adjuvants.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker, e.g. myosin or actin or the expression of a gene or protein, e.g. a cell surface marker. In another aspects, the substantially homogenous population have a decreased (e.g., less than about 95%, or alternatively less than about 90%, or alternatively less than about 80%, or alternatively less than about 75%, or alternatively less than about 70%, or alternatively less than about 65%, or alternatively less than about 60%, or alternatively less than about 55%, or alternatively less than about 50%) of the normal level of expression than the wild-type counterpart cell or tissue.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention. The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the V_{L}, V_{H}, C_{L} and CH, domains; a F(ab′)2 fragment, a bivalent fragment comprising two Fab fragments linked by a disulfide bridge at the hinge region; a Fd fragment consisting of the V_{H} and C_{H}, domains; a Fv fragment consisting of the V_{L} and V_{H} domains of a single arm of an antibody, a dAb fragment (Ward, E. S. et al. (1989) Nature 341:544-546), which consists of a V_{H} domain; and an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)). (Bird, R. E. et al. (1988) Science 242:423-426; Huston, J. S. et al. (1988) Proc. Natl. Acad Sci. USA 85:5879-5883). Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention. The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. It also includes in some aspects, antibody variants, polyclonal antibodies, human antibodies, humanized antibodies, chimeric antibodies, antibody derivatives, a bispecific molecule, a multispecific molecule, a heterospecific molecule, heteroantibodies and human monoclonal antibodies.

Examples of binding fragments encompassed within the term “antigen binding portion” of an antibody include a Fab fragment, a monovalent fragment consisting of the V_{L}, V_{H}, C_{L} and CH, domains; a F(ab′)2 fragment, a bivalent fragment comprising two Fab fragments linked by a disulfide bridge at the hinge region; a Fd fragment consisting of the V_{H} and C_{H}, domains; a Fv fragment consisting of the V_{L} and V_{H} domains of a single arm of an antibody, a dAb fragment (Ward, E. S. et al. (1989) Nature 341:544-546), which consists of a V_{H} domain; and an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)). (Bird, R. E. et al. (1988) Science 242:423-426; Huston, J. S. et al. (1988) Proc. Natl. Acad Sci. USA 85:5879-5883). Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “antibody variant” is intended to include antibodies produced in a species other than a mouse. It also includes antibodies containing post-translational modifications to the linear polypeptide sequence of the antibody or fragment. It further encompasses fully human antibodies.

The term “antibody derivative” is intended to encompass molecules that bind an epitope as defined above and which are modifications or derivatives of a native monoclonal antibody of this invention. Derivatives include, but are not limited to, for example, bispecific, multispecific, heterospecific, tri specific, tetraspecific, multispecific antibodies, diabodies, chimeric, recombinant and humanized.

The term “bispecific molecule” is intended to include any agent, e.g., a protein, peptide, or protein or peptide complex, which has two different binding specificities. The term “multispecific molecule” or “heterospecific molecule” is intended to include any agent, e.g. a protein, peptide, or protein or peptide complex, which has more than two different binding specificities.

The term “heteroantibodies” refers to two or more antibodies, antibody binding fragments (e.g., Fab), derivatives thereof, or antigen binding regions linked together, at least two of which have different specificities.

The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., Cₘ, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

The terms “monoclonal antibody” or “monoclonal antibody composition” as used herein refer to a preparation of antibody molecules of single molecular composition. A monoclonal antibody composition displays a single binding specificity and affinity for a particular epitope.

A “human monoclonal antibody” refers to antibodies displaying a single binding specificity which have variable and constant regions derived from human germline immunoglobulin sequences.

A “host cell” include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escherichia coli, Salmonella enterica and Streptococcus gordonii. In one embodiment, the host cell is E. coli. The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

The terms “culture” or “culturing” refer to the in vitro propagation of cells or organisms on or in media of various kinds. It is understood that the descendants of a cell grown in culture may not be completely identical (i.e., morphologically, genetically, or phenotypically) to the parent cell.

A “subject” of diagnosis or treatment is a cell or a mammal, including a human. Non-human animals subject to diagnosis or treatment include, for example, simians, murines, guinea pigs, canines, such as dogs, leporids, such as rabbits, livestock, such as bovine or porcine, sport animals, and pets.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms. In one aspect, the treatment is to induce an immune response. As used herein, the term “induce an immune response” intends recognition of the IgG by immune cells which then become activated and attack the labeled cancer cells with great specificity, with little or no inflammation. Methods to measure and determine if an immune response been induced activation of the immune cells are known in the art.

The term “suffering” as it related to the term “treatment” refers to a patient or individual who has been diagnosed with or is predisposed to infection or a disease incident to infection. A patient may also be referred to being “at risk of suffering” from a disease because of active or latent infection. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic cells and compositions of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the composition that is effective to achieve a serum level commensurate with the concentrations found to be effective in vitro. Determination of these parameters is well within the skill of the art. These considerations, as well as effective formulations and administration procedures are well known in the art and are described in standard textbooks.

The term “administration” shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

A “control” is an alternative subject or sample used in an experiment for comparison purpose. A control can be “positive” or “negative”. For example, where the purpose of the experiment is to determine a correlation of a mutated allele with a particular phenotype, it is generally preferable to use a positive control (a sample from a subject, carrying such mutation and exhibiting the desired phenotype), and a negative control (a subject or a sample from a subject lacking the mutated allele and lacking the phenotype).

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

A neoplasm is an abnormal mass or colony of cells produced by a relatively autonomous new growth of tissue. Most neoplasms arise from the clonal expansion of a single cell that has undergone neoplastic transformation. The transformation of a normal to a neoplastic cell can be caused by a chemical, physical, or biological agent (or event) that directly and irreversibly alters the cell genome. Neoplastic cells are characterized by the loss of some specialized functions and the acquisition of new biological properties, foremost, the property of relatively autonomous (uncontrolled) growth. Neoplastic cells pass on their heritable biological characteristics to progeny cells.

The past, present, and future predicted biological behavior, or clinical course, of a neoplasm is further classified as benign or malignant, a distinction of great importance in diagnosis, treatment, and prognosis. A malignant neoplasm manifests a greater degree of autonomy, is capable of invasion and metastatic spread, may be resistant to treatment, and may cause death. A benign neoplasm has a lesser degree of autonomy, is usually not invasive, does not metastasize, and generally produces no great harm if treated adequately.

Cancer is a generic term for malignant neoplasms. Anaplasia is a characteristic property of cancer cells and denotes a lack of normal structural and functional characteristics (undifferentiation).

A tumor is literally a swelling of any type, such as an inflammatory or other swelling, but modern usage generally denotes a neoplasm. The suffix “-oma” means tumor and usually denotes a benign neoplasm, as in fibroma, lipoma, and so forth, but sometimes implies a malignant neoplasm, as with so-called melanoma, hepatoma, and seminoma, or even a non-neoplastic lesion, such as a hematoma, granuloma, or hamartoma. The suffix “-blastoma” denotes a neoplasm of embryonic cells, such as neuroblastoma of the adrenal or retinoblastoma of the eye.

“Suppressing” tumor growth indicates a growth state that is curtailed compared to growth without any therapy. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and “suppressing” tumor growth indicates a growth state that is curtailed when stopping tumor growth, as well as tumor shrinkage.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Non-limiting examples of embryonic stem cells are the HES2 (also known as ES02) cell line available from ESI, Singapore and the H1 (also know as WA01) cell line available from WiCells, Madison, Wis. Pluripotent embryonic stem cells can be distinguished from other types of cells by the use of marker including, but not limited to, Oct-4, alkaline phosphatase, CD30, TDGF-1, GCTM-2, Genesis, Germ cell nuclear factor, SSEA1, SSEA3, and SSEA4.

A “mesenchymal stem cell” or MSC, is a multipotent stem cell that can differentiate into a variety of cell types. The designation MSC also refers to the term “marrow stromal cell”. Cell types that MSCs have been shown to differentiate into in vitro or in vivo include osteoblasts, chondrocytes, myocytes, and adipocytes. Mesenchyme is embryonic connective tissue that is derived from the mesoderm and that differentiates into hematopoietic and connective tissue, whereas MSCs do not differentiate into hematopoietic cells. Stromal cells are connective tissue cells that form the supportive structure in which the functional cells of the tissue reside. While this is an accurate description for one function of MSCs, the term fails to convey the relatively recently-discovered roles of MSCs in repair of tissue. Applicants have described methods to isolate, propagate, and genetically engineer marrow stromal cells/mesenchymal stem cells (MSC) for over two decades (reviewed in Nolta, Genetic Engineering of Mesenchymal Stem Cells, Springer 2006). Methods to isolate such cells, propagate and differentiate such cells are known in the technical and patent literature, e.g., U.S. Patent Application Publication Nos. 2007/0224171, 2007/0054399, 2009/0010895, which are incorporated by reference in their entirety.

A clone or “clonal population” is a line of cells that is genetically identical to the originating cell; in this case, a stem cell. A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a farther stage of cell differentiation. Progenitor cells are often found in adult organisms, they act as a repair system for the body. Examples of progenitor cells include, but are not limited to, satellite cells found in muscles, intermediate progenitor cells formed in the subventricular zone, bone marrow stromal cells, periosteum progenitor cells, pancreatic progenitor cells and angioblasts or endothelial progenitor cells. Examples of progenitor cells may also include, but are not limited to, an ependymal cell and a neural stem cell from the forebrain lateral ventricle (LV).

As used herein, the term “codon-optimized” itends a polypeptide that has been codon optimized. Codon optimization is a technique to improve the protein expression in living organism by increasing the translational efficiency of gene of interest. Methods to optimize expression by this technique are known in the art. See, eg., the web page openwetware.org/wiki/Synthetic_Biology:Tools, last accessed on Dec. 15, 2014, for tools and information related to codon optimization.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Clonal proliferation” refers to the growth of a population of cells by the continuous division of single cells into two identical daughter cells and/or population of identical cells.

As used herein, the “lineage” of a cell defines the heredity of the cell, i.e., its predecessors and progeny. The lineage of a cell places the cell within a hereditary scheme of development and differentiation.

A “derivative” of a cell or population of cells is a daughter cell of the isolated cell or population of cells. Derivatives include the expanded clonal cells or differentiated cells cultured and propagated from the isolated stem cell or population of stem cells. Derivatives also include already derived stem cells or population of stem cells.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, a “pluripotent cell” defines a less differentiated cell that can give rise to at least two distinct (genotypically and/or phenotypically) further differentiated progeny cells. In another aspect, a “pluripotent cell” includes a Induced Pluripotent Stem Cell (iPSC) which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e., Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e., c-myc and L-myc; the family of Nanog genes, i.e., OCT4, NANOG and REX1; or LIN28. Examples of iPSCs are described in Takahashi, K. et al. (2007) Cell advance online publication 20 Nov. 2007; Takahashi, K. & Yamanaka, S. (2006) Cell 126: 663-76; Okita, K. et al. (2007) Nature 448:260-262; Yu, J. et al. (2007) Science advance online publication 20 Nov. 2007; and Nakagawa, M. et al. (2007) Nat. Biotechnol. Advance online publication 30 Nov. 2007.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A “population of cells” intends a collection of more than one cell that is identical (clonal) or non-identical in phenotype and/or genotype.

### MODES FOR CARRYING OUT THE INVENTION

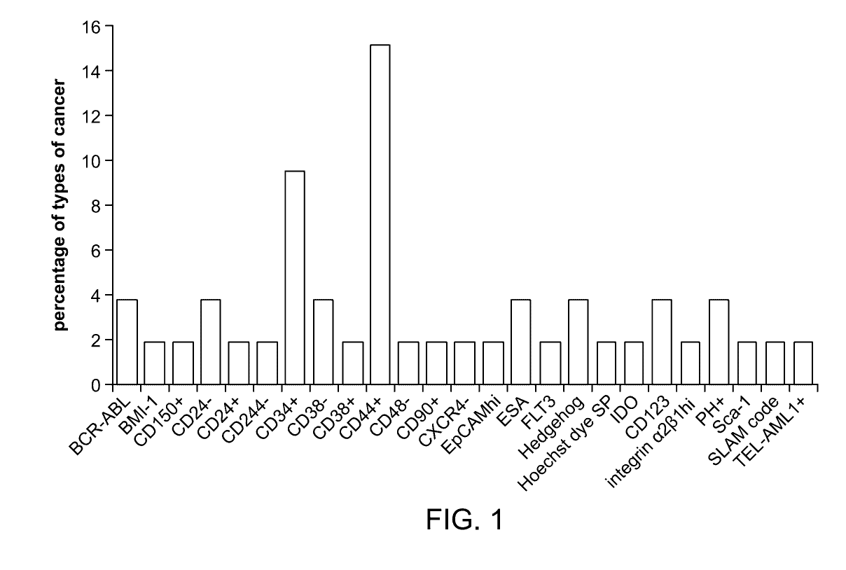

With the discovery of tumorigenic cancer stem cells (CSC), the cell surface glycoprotein cluster of differentiation 44 (CD44) was quickly identified as a CSC cell surface marker. CD44 appears to be one of the most commonly found cell surface markers on CSC of numerous forms of cancers, including various types of carcinoma, neuroblastoma, and leukemia (FIG. 1). Until recently, the specific form of CD44 was not understood to be of great importance in distinguishing between the variant (CD44v of which there are several) and standard (CD44s) forms. The normal physiological function of CD44 is to help cells communicate with each other and their environment through cell-cell and cell-extracellular matrix interactions. Prior to its vast infamy as a CSC marker, CD44 was known more peaceably as the receptor for the extracellular matrix protein hyaluronan and for its participation in normal cellular functions that are incidentally still being revealed such as in hematopoiesis (Sackstein, R. (2011) Current Opinion in Hematology 18:239-248) and dental mineralization (Chen, K. L. et al. (2013) Journal of Endodontics 39:351-356). Its role in cancer and metastasis has highlighted that CD44 also interacts with osteopontin (Rao, G. et al. (2013) Clinical Cancer Research 19:785-797), various collagens, and matrix metalloproteinases (Xu, Y. P. et al. (2003) Journal of Zhejiang University Science 4:491). The variant forms are found more routinely in environments where there are high levels of mitogenic signaling, such as that of malignant tumors (Ponta, H. et al. (2003) Nature Reviews Molecular Cell Biology 4:33-45). It is now clear that CD44v6 is the variant isoform involved in many aggressive traits of cancer, interacting with pathways that increase differentiation potential (Bendardaf, R. et al. (2005) Oncol. Rep. 13:831-835), cellular motility (Wang, H. et al. (2013) International Journal of Cancer), and increased cell survival (Jung, T. et al. (2011) Journal of Biological Chemistry 286:15862-15874). The strong utility of CD44v6 in the invasive and metastatic agenda of cancer cells suggests that it is not a redundant or easily replaceable protein in cancer.

The innate ability and actions of mesenchymal stem (or stromal) cells (MSC) to home to sites of hypoxia and inflammation including the tumor bed, is extensively described in the literature (Doucette, T. et al. (2011) Neoplasia 13:716-725; Hong, H. S. et al. (2012) Archives of Pharmacal Research 35:201-211; Reagan, M. R. et al. (2011) Stem Cells 29:920-927; Pulukuri, S. M. et al. (2010) Molecular Cancer Research 8:1074-1083) and shown herein as well. Although several pathways involving inflammatory cytokines (e.g., IL-6) have been implicated to play roles in the enhanced migratory signaling of MSC trafficking, the precise mechanisms through which MSC are recruited to these sites of inflammation and hypoxia are not fully understood.

Applicants have shown that by harnessing the intrinsic properties of MSC to home to the tumor bed and increase expression of CCL5 within the tumor environment, tumor targeting can be achieved. Rather than attempting to attack the bulk tumor, this approach seeks out and flags tumorigenic cells presenting them to the immune system, while stimulating complement and cytotoxic immune cells, and being highly effective against metastases even before they become clinically relevant.

### Compositions

With the preceding concepts in mind, this disclosure provides an isolated polynucleotide encoding an in vivo tumor targeting antibody, the polynucleotide comprising, or alternatively consisting of, or yet consisting of, a promoter element that drives expression of the C—C motif ligand 5 (“a CCL5 promoter”) operatively linked to a polynucleotide encoding a fusion polypeptide, the fusion polypeptide comprising the Fc region of a human antibody selected from the group: IgG1, IG2, IgG3, IgG4, IgA1, IgA2 or IgM and a ScFv region of an anti-CD44, in one aspect a CD44 variant, e.g., CD44v6 (CD44v6) polypeptide. In a further aspect, the polynucleotide further comprises, or alternatively consists essentially of, or yet further consists of a polynucleotide encoding a hinge of an antibody hinge region, e.g., a human hinge region. In one aspect, the human antibody is a human IgG1 antibody. In a further aspect, the IgG1 polynucleotide comprises any one of SEQ ID NOS. 3 or 4, or an equivalent of each thereof. In one aspect the polynucleotide is RNA. In another aspect the polynucleotide is DNA.

An example of a promoter element is the CCL5 promoter polynucleotide that comprises, or alternatively consisting essentially of, or yet further consisting of, nucleotides from about 2183 to about 3554 of SEQ ID NO. 1, a biologically active fragment or an equivalent thereof, that drives expression of a polynucleotide. As used herein, the term “CCL5 promoter element” intends a full length or fragment of a promoter, as disclosed herein, as well as equivalents thereof, as defined herein. Any fragment of the disclosed CCL5 promoter is intended within the scope of this disclosure, as long as the polynucleotide promotes expression of the polynucleotide encoding the fusion polypeptide.

In one aspect, the isolated polynucleotide as described above, further comprises, or alternatively consisting essentially of, or yet consisting of, a polynucleotide encoding the signal sequence of a secreted protein. In one aspect, the signal sequence is any type II signal sequence, examples of which are known in the art. A non-limiting example of a type II signal sequence is the signal sequence of the secreted protein is Interleukin-2 (“IL-2”) such as the polynucleotide shown in SEQ ID NO. 1, from about nucleotides 3560 to about 3647, and biological equivalents thereof. The polynucleotide is DNA or RNA.

In another aspect, the polynucleotide encoding the anti-CD44v6 polypeptide comprises, or alternatively consists essentially of, or yet further consists of, a polynucleotide comprising, or alternatively consisting of, or yet consisting of, from about nucleotides 3648 to about 4049 of SEQ ID NO. 1, a fragment having the same or similar biological activity or a biological equivalent thereof or alternatively, SEQ ID NO. 2 a fragment having the same or similar biological activity or a biological equivalent thereof. The polynucleotide is DNA or RNA.

In a further aspect, the isolated polynucleotide as described above, further comprises, or alternatively consisting of, or yet consisting of, a polynucleotide encoding a membrane targeting signal sequence. In one aspect, the membrane targeting signal sequence comprises, or alternatively consists essentially of, or yet further consisting of, a polynucleotide comprises nucleotides from about 6023 to about 6083 of SEQ ID NO.: 1, a fragment having the same or similar biological activity or a biological equivalent thereof. The polynucleotide is DNA or RNA.

In another aspect, the isolated polypeptide as described above further comprises, or alternatively consisting of, or yet further consisting of, a polynucleotide encoding a detectable label or a detectable label. The polynucleotide encoding a detectable label is DNA or RNA. Non-limiting examples of the detectable label is one or more of a mCherry Red Fluorescent Protein, tdTomato, a green fluorescent protein (GFP), a yellow fluorescent protein (YFP) and a cyan fluorescent protein (CFP). A non-limiting example of a polynucleotide encoding mCherry RFP is provided in SEQ ID NO. 1, from about nucleotides 6101 to about 6793 or a biologically active fragment thereof or an equivalent thereof. In a further aspect, the polynucleotide encoding the detectable protein, e.g., mCherry RFP, is under the control of an IRES polynucleotide. A non-limiting example of an IRES polynucleotide is provided in SEQ ID NO. 1, from about nucleotides 5451 to about 6022, or a biologically active fragment thereof or an equivalent thereof.

In another aspect, the isolated polynucleotide as disclosed above, further comprises, or alternatively consists essentially of, or yet further consists of, an enhancer, e.g., a Woodchuck Hepatitis Virus Postranscriptional Regulatory Element (WPRE) polynucleotide. A non-limited example of an WPRE polynucleotide comprises, or alternatively consists essentially of, or consists of, SEQ ID NO: 13, alternatively comprising, or alternatively consisting essentially of, or yet further consisting of, from about nucleotide of 6807 to about 7398 of SEQ ID NO.: 1, or a biologically active fragment thereof or a biological equivalent thereof. The polynucleotide is DNA or RNA.

The isolated polynucleotide of any one of claims 1 to 10, further comprising a 5′ LTR and a 3′ LTR operatively linked to the isolated polynucleotide. A non-limiting example of a 5′ LTR polynucleotide is provided in SEQ ID NO. 1, or one comprising, or alternatively consisting essentially of, or yet consisting of, from about nucleotides 1 to about 635, or a biologically active fragment thereof or an equivalent thereof. A non-limiting example of a 3′ LTR polynucleotide is provided in SEQ ID NO. 1, from about nucleotides 7601 to about 8237, a biologically active fragment thereof or an equivalent thereof.

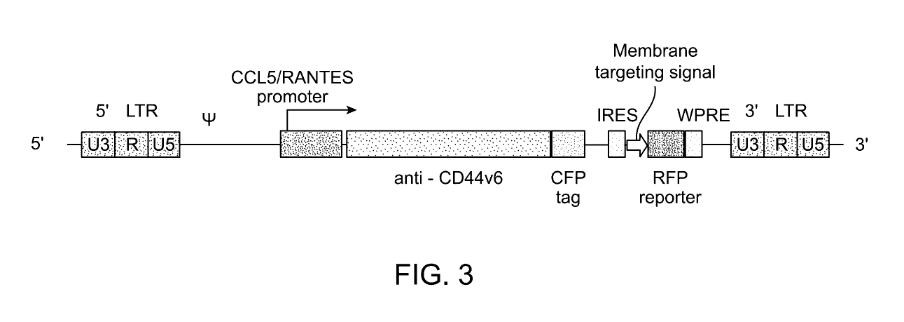

In a further aspect, the polynucleotide encoding the tumor targeting antibody is schematically shown in FIG. 3 and includes equivalents thereof. In a further aspect, the isolated polynucleotide encoding the fusion polypeptide, or consists essentially of, or consists of SEQ ID NO. 1, or an equivalent thereof. The polynucleotide is DNA or RNA. Equivalents and biologically active fragments of the components of the polynucleotide are described above.

The isolated polynucleotides can be inserted and contained within a vector for expression or replication of the polynucleotide. This, this disclosure provides the isolated polynucleotide as described above in a vector, such as a plasmid or viral vector. In one aspect, the vector is a plasmid or a lentiviral vector.

The isolated polynucleotides can be inserted and contained within a host cell for expression or replication of the polynucleotide, or for delivery of the fusion polypeptide in vivo. This, this disclosure provides the isolated polynucleotide as described above in a host cell, alone or within a vector, such as a plasmid or viral vector, e.g., lentiviral vector. The isolated host cell can be a prokaryotic or a eukaryotic cell. A non-limiting examples of a prokaryotic cells include E. coli. Non-limiting examples of eukaryotic cells include mammalian cells, yeast cells, human cells, murine cells. Prokaryotic and eukaryotic cells are commercially available from vendors such as the American Type Culture Collection (ATCC). In one aspect, the host cell is a stem cell, e.g., a human stem cell. In a particular embodiment, the human stem cell is a mesenchymal stem cell.

The host cell can be cultured and grown under favorable conditions for the expression of the fusion peptide. Thus, this disclosure also provides a method for expressing or producing the fusion peptide, in vitro or in vivo, by growing the host cell under conditions favorable to expression. In one aspect, the peptide is secreted from the host cell. In another aspect, the fusion peptide is post-translationally modified. In a yet further aspect, any peptide produced by the vector and/or host cell system is isolated or separated from the host cell. In a further aspect, the disclosure provides a clonal population or a substantially homogenous (i.e., greater than 50%, or alternatively greater than 60%, or alternatively greater than 70%, or alternatively greater than 75%, or alternatively greater than 80%, or alternatively greater than 85%, or alternatively greater than 90%, or alternatively greater than 95%, or alternatively, greater than 98%) population of host cells transfected or transduced with the polynucleotide and/or vector.

In one aspect, the isolated host cell as described above, is an isolated stem cell. The stem cell can be an embryonic, iPSC, or adult or somatic stem cell. In one aspect, the stem cells is an adult stem cell, such as a mesenchymal stem cell. The disclosure also provides a population of stem cells, that can be substantially homogenous or clonal, and can include stem cells differentiated from a parental stem cell of this disclosure. Methods for growing, differentiating and clonally expanding stem cells are known in the art.

In a further aspect, the isolated host cell is a mesenchymal stem cell of mammalian origin, e.g., bovine, ovine, canine, equine, murine, simian, feline or human origin. In a further aspect the mesenchymal stem cells is characterized by one or more markers of the group: CD73, CD166, nucleostemin, CD44, CD45, CD90, CD45RO, CD105, CD54, CD49a, CD49e, CD51, CD29, CD56, Sca-1, SCF R/c-kit, SSEA-4, STRO-1, TfR, CD106, vimentin, and/or having the capacity to differentiate into an adipose cell, a bone cell, a cartilage cell, and a muscle cell or tissues comprising one or more of the cells. Further provided are isolated clonal population of such cells, or a population of cells that are substantially homogenous, e.g., about 75%, or about 80%, or about 85%, or about 90%, or about 95% or about 97% or about 98% substantially homogenous. When the cells are stem cell, this disclosure also provides differentiated cells or populations of cells that the product of culturing the stem cells under conditions that favor differentiation of the cell.

Compositions comprising, consisting essentially of, or yet further consisting of: 1) a carrier and 2) one or more of an isolated polynucleotide as described herein, the fusion polypeptide, a translationally modified fusion polypeptide, polynucleotides encoding such, the vector as described herein, the isolated host cell as described herein, or the population of cells, as described herein, are further provided. The populations of compositions can be substantially homogeneous. In one aspect, the carrier is a pharmaceutically acceptable carrier.

### Methods and Utility

The compositions describe above are useful in methods for delivering a fusion polypeptide to a tumor cell expressing CD44 marker, including all isoforms of CD44s or CD44v, e.g., CD44v6, comprising, or alternatively consisting essentially of, or yet further consisting of, contacting the tumor cell with an effective amount of the isolated host cell as described above, in particular when the host cell is a mesenchymal stem cell or a population of mesenchymal stem cells. Non-limiting examples of tumors include carcinomas, sarcinomas and leukemia, more particularly breast cancer, pancreatic cancer, prostate cancer and lung cancer. The contacting (meaning bringing into immediate or close proximity thereto) can be performed, in vitro, ex vivo or in vivo. When the cells contacting is in vivo, the cells can be autologous or allogeneic, and the cell or population of cells are administered to a patient or subject, such as a human patient.

The compositions describe above are useful in methods for one or more of: inducing an immune response or for inhibiting the growth of a tumor, each in a subject in need thereof, comprising, or alternatively consisting essentially of, or yet further consisting of, administration of an effective amount of the isolated host cell as described above, in particular when the host cell is a mesenchymal stem cell or a population of mesenchymal stem cells. In one aspect, the subject is suffering from cancer. In a further aspect, the subject is suffering from a cancer or has a tumor that expresses the CD44 marker, e.g., a CD44 variant such as CD44v6. Non-limiting examples of tumors include carcinomas, sarcinomas and leukemia, more particularly breast cancer, pancreatic cancer, prostate cancer and lung cancer. In another aspect, the subject is a mammal, e.g., a simian, a bovine, a canine, a feline, an equine or a human. In these methods, the isolated host cell is autologous or allogeneic to the subject.

Any appropriate method of administration can be utilized, e.g, intravenous injection, perfusion or infusion.

The methods can further comprise, or alternatively consist essentially of, or yet further consist of, administration of an effective amount of an anti-tumor therapy or an immune supplement. Non-limiting examples, of anti-tumor therapies include surgical resection, chemotherapy or radiation therapy.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

The agents and compositions for use in the methods of this invention can be concurrently or sequentially administered with other anticancer agents. Non-limiting examples of administration include by one or more method comprising transdermally, urethrally, sublingually, rectally, vaginally, ocularly, subcutaneous, intramuscularly, intraperitoneally, intranasally, by inhalation or orally.

Thus, routes of administration applicable to the methods of the invention include intravenous, intranasal, intramuscular, urethrally, intratracheal, subcutaneous, intradermal, topical application, rectal, nasal, oral, inhalation, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery, include systemic or localized routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the inhibiting agent. Where systemic delivery is desired, administration typically involves invasive or systemically absorbed topical or mucosal administration of pharmaceutical preparations.

### In Vivo Testing

As is apparent to the skilled artisan, a marker expressing cell line, e.g., a tumor cell line expressing or overexpressing CD44v6, is transplanted subcutaneously into immunodeficient mice, or mice derived from immunodeficient mice (e.g., humanized mice). Initial therapy can be infusion or direct intratumoral injection. The expected result is that an immune response leading to antitumor activity by the compositions of this disclosure.

### Companion Diagnostics

This disclosure also provides a diagnostic method and tools to identify patients that are more likely to respond to the therapeutic methods of this disclosure. In one aspect, the invention is a method for identifying responsiveness to the above-noted by assaying a suitable patient sample from a patient suffering from cancer or tumor for expression of the CD44 marker. In one aspect, the marker is a CD44 variant, e.g., CD44v6. Patients having low gene expression of the CD44 marker are less likely to respond to the therapy of this disclosure.

As used herein, responsiveness is any positive clinical or sub-clinical response, such as reduction in tumor load or size, increase in time to tumor progression, increase in progression free survival or increase in overall survival.

To practice this method, the sample is a patient sample containing a non-metastatic or metastatic tumor cell, non-metastic tumor tissue, metastatic tumor cell or metastatic tumor tissue or a blood cell, e.g., a peripheral blood leukocyte (PBL).

These methods are not limited by the technique that is used to identify the expression or gene expression levels or the marker. Suitable methods include but are not limited to the use of hybridization, antibodies, polymerase chain reaction (PCR) analysis, protein expression, gene chips and software for high throughput analysis. Additional genes can be assayed and used as negative controls.

For example, one method of this invention can be practiced by: (a) obtaining a suitable sample of the patient's tumor or other suitable sample; (b) isolating mRNA or protein from the sample; (c) determining the amount of CD44mRNA in the sample or determining if CD44 is expressed by the tumor cell; (d) comparing the amount of CD44 mRNA from step (c) to an amount of mRNA of an internal control gene such as (3-actin mRNA or another internal control to determine the difference between the amplified sample and internal control gene; and (e) comparing the difference of step (d) to a predetermined threshold level for CD44 gene expression, thereby identifying the expression level as either high or low. In one aspect, the marker is a CD44 variant, e.g., CD44v6.

To practice this method, the sample is a patient sample containing a non-metastatic or metastatic tumor cell, non-metastic tumor tissue, metastatic tumor cell or metastatic tumor tissue or a blood cell, e.g., a peripheral blood leukocyte (PBL).

After a patient has been identified as likely responsive based on the expression levels, the method may further comprise, or alternatively consist essentially of, or yet further consist of, administration or delivery of an effective amount of a therapy of this disclosure.

In another aspect, the invention is a method for identifying and selecting a therapy comprising a therapy of this disclosure by assaying a suitable patient sample from a patient suffering from a solid malignant tumor for the gene expression level or expression of CD44. Tumors expressing CD44 are more likely than not to be suitably treated by the therapy of this disclosure and therefore, therapy should be selected.

To practice this method, the sample is a patient sample containing the non-metastic tumor cell, non-metastic tumor tissue, metastatic tumor cell or metastatic tumor tissue. These methods are not limited by the technique that is used to identify the expression of CD44. Suitable methods include but are not limited to the use of hybridization, antibodies, PCR analysis, protein expression, gene chips and software for high throughput analysis. Additional genes can be assayed and used as negative controls.

To practice this method, the sample is a patient sample containing a non-metastatic or metastatic tumor cell, non-metastic tumor tissue, metastatic tumor cell or metastatic tumor tissue or a blood cell, e.g., a peripheral blood leukocyte (PBL).

Suitable methods include but are not limited to the use of hybridization, antibodies, PCR analysis, protein expression, gene chips and software for high throughput analysis. Additional genes can be assayed and used as negative controls.

This invention also provides a panel, a kit, software, support or gene chip for patient sampling and performance of the methods of this invention. The kits contain gene chips, probes or primers that can be used to amplify and/or determining the expression and/or expression level of CD44 or a variant thereof, e.g., CD44v6. In an alternate embodiment, the kit contains antibodies or other polypeptide binding agents that are useful to identify the expression or expression levels of CD44. Instructions for using the materials to carry out the invention are further provided alone or in combination with instructions for administration of a therapy as described herein.

### Kits

This disclosure also provides kits comprising, or alternatively consisting essentially of, or yet further consisting of, one or more of a composition as described herein, and instructions for use. They may be used in the preparation of a medicament for the treatment of a disease or condition as described herein.

### Materials and Methods

### Animals

NOD-SCID IL2Rγ−/− (NSG) and humanized NSG mice, xenografted with MDA-MB-231 cancer cells or other cancer cells isolated from fresh patient samples obtained under UCD IRB approval, were maintained at the UCDMC Institute for Regenerative Cures Vivarium with standard mouse chow and water ad libitum, under strict barrier facility Standard Operating Procedures (SOPs). GFP-GFP/LUC expression (the cells had constitutive expression of both the GFP and the Luciferase protein) or engineered MSC were intravenously introduced to animals after flank tumors had developed, by tail-vein injection. All protocols were performed with approval by the UC Davis Institutional Animal Care and Use Committee (IACUC).

### Cell Culture

The metastatic cancer cell line MDA-MB-231 (ATCC) and human bone marrow-derived mesenchymal stem (stromal) cells (MSC) as well as green fluorescent protein-expressing MSC (GFP⁺MSC) were cultured at 5% CO2/3% 02. Medium for MDA-MB-231 cells was DMEM (4500 mg/L glucose) supplemented with 1% L-glutamine (Gibco Invitrogen), 1% penicillin/streptomycin, 10% FBS. Medium for MSC and GFP⁺MSC was composed of α-MEM supplemented 1% L-glutamine, 1% penicillin/streptomycin, and 20% FBS.

### Measurement of CCL5/RANTES

MDA-MB-231 cells were loaded with Cell Trace CFSE (Invitrogen) according to manufacturer's instruction for adherent cells. Loading of the dye was confirmed by fluorescence microscopy to ensure all of the cells were loaded. Co-cultures of bone marrow-derived MSC and CFSE-loaded MDA-MB-231 cells were set up at an approximately 30:70 ratio. MSC were gradually adjusted to MDA-MB-231 media prior to co-culture. Media consisting of DMEM supplemented with 10% FBS, 1% L-glutamine, and 1% penicillin/streptomycin was changed 24 hours following plating, and replaced every 24 hours thereafter as supernatants were collected. Supernatants were collected every day for 6 days and immediately frozen and stored at −20° C. Secreted CCL5 was quantified by cytometric bead array (BD Biosciences) according to the manufacturer's instructions, using a Beckman Coulter FC500 flow cytometer. MSC and cancer cells were separated by FACS (fluorescence activated cell sorting) using an InFlux Beckton Dickinson-Cytopeia Cell Sorter, operated by UC Davis Flow Cytometry Core Facilities. RNA was immediately isolated following FACS using RNA Stat reagent. An aliquot was taken for reverse transcription to cDNA, while the remainder was immediately stored at −80° C. Expression of CCL5 was measured by quantitative reverse transcriptase PCR (qRT-PCR) at day 0 through day 5 using the following primers.

FWD:
(SEQ ID NO: 11)
5′ TGCAGAGGATCAAGACAGCA 3′
REV:
(SEQ ID NO: 12)
5′ GAGCACTTGCCACTGGTGTA 3′

### Under-Agarose Cell Migration Assays

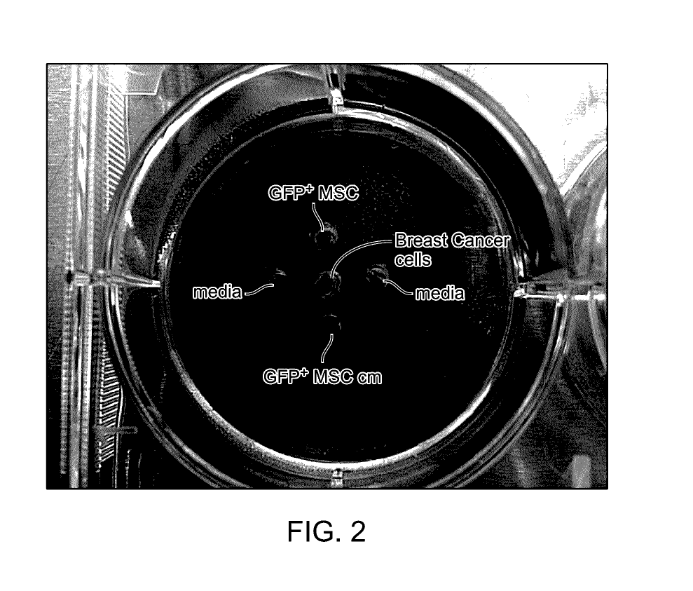

The protocol used was adapted from Heit and Kubes, 2003 (Heit, B. et al. (2003) Science's STKE 2003(170):PL5) with minor changes. Briefly, media consisting of 33% HBSS media, 53% DMEM^{hi-glucose}, 13% FBS, 1% penicillin/streptomycin was mixed in a 30:10 ratio with 48% w/v ultra-pure agarose (Invitrogen) dissolved in milliQ-filtered water, immediately applied to tissue culture dishes, and allowed to solidify under sterile conditions at room temperature. Once solidified, a p1000 micropipette was used to create patterned wells as shown in FIG. 2. Tissue culture dishes were allowed to equilibrate at 5% CO2/3% O₂ and 37° C. before loading. Cells were loaded first at approximately 10,000 cell per well in 2-7 μL and allowed to settle for approximately 8 hours. In some experiments MDA-MB-231 cells were incubated with CD44v6 blocking antibody (Damm, S. K. P. et al. (2010) Journal of Investigative Dermatology 130:1893-1903) (BD Biosciences) or CCR5 blocking antibody (Honczarenko, M. et al. (2002) Blood 100:2321-2329) (R&D) for 30 minutes at 37° C. prior to loading into wells. Chemokines were mixed to their final concentration in appropriate MSC or MDA-MB-231 media and loaded. Final concentrations were as follows; osteopontin (BD Biosciences) 11 μg/ml, interleukin-6 (BD Biosciences) 50 ng/ml, CCL5/RANTES (Millipore) based on previously described concentrations found in patient tumors (Yamada, D. et al. (2013) European Journal of Cancer). Loaded dishes were then incubated under the same conditions and visualized with a Nikon Ti-U microscope, or incubated at 5% CO2/16% O₂ and 37° C. in a BioStation IM (Nikon) for timelapse recording.

### Lentiviral Vector Design and Preparation

A lentiviral vector background was used with a ubiquitous EF1α promoter into which were cloned the nucleotide sequence corresponding to the amino acid sequence of a tested single chain antibody against CD44v6 (Chen, Y. et al. (2010) Cancer Immunology Immunotherapy 59:933-942) (DNA2.0), in between sequences comprising an IL2 signal sequence and that of a human IgG1 engineered Fc region comprising the CH2 and CH3 domains of the IgG heavy chain and hinge region (InvivoGen). Following this was inserted the nucleotide sequences encoding an AmCyan fluorescent protein (CFP) to create a tagged a fusion protein antibody. To facilitate visualization that the gene for the antibody is being expressed, a mCherry sequence was inserted behind an internal ribosomal entry site (IRES) targeted to the cellular membrane with a membrane targeting sequence (mts) (Clontech). For tumor-specific expression of the antibody, the EF1α promoter was swapped out for a CCL5 promoter cut out from another plasmid (Xactogen) (FIG. 3). Bench preparation and viral packaging of lentiviral vectors were performed by the UC Davis Stem Cell Program Vector Core. Multiplicity of infection (MOI) was optimized by titer determination directly on bone marrow-derived MSC. MSC were transduced at about 30% confluence in transduction medium made up of culture medium as above supplemented with protamine sulfate at an MOI of 10 for 48 hours with incubation at 5% CO2/3% 02 and 37° C. After 48 hours the transduction medium was removed, cells were rinsed briefly with PBS and fresh MSC-medium was added. Medium was replaced every 1-2 days.

MSC transduced with vector having the CCL5 promoter were cultured for 3 days then media was progressively switched to MDA-MB-231 (non-conditioned) media as follows: day 1-25% MDA-MB-231 media/75% MSC media; day 2-50% MDA-MB-231 media/50% MSC media; day 3 75% MDA-MB-231 media/25% MSC media; day 4 100% MDA-MB-231 media. Then days 5-7 with 100% conditioned media, with visualization by fluorescence and time-lapse video recording at days 5 through 7.

### Results

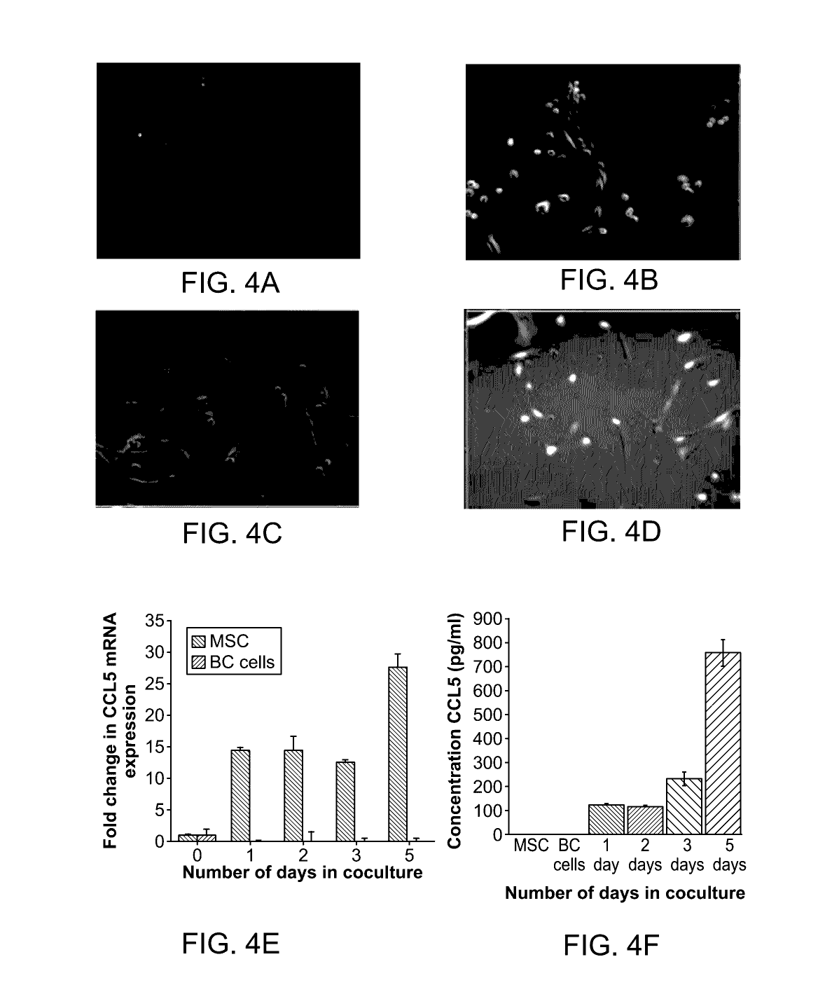

Co-cultures of MSC with MDA-MB-231 cells resulted in a significant increase in secreted CCL5 measured in the cell culture media. After separating the cells by FACS, qRT-PCR revealed that the increase in CCL5 secretion was due to increased expression in MSC, compared to the cancer cells (FIG. 4).

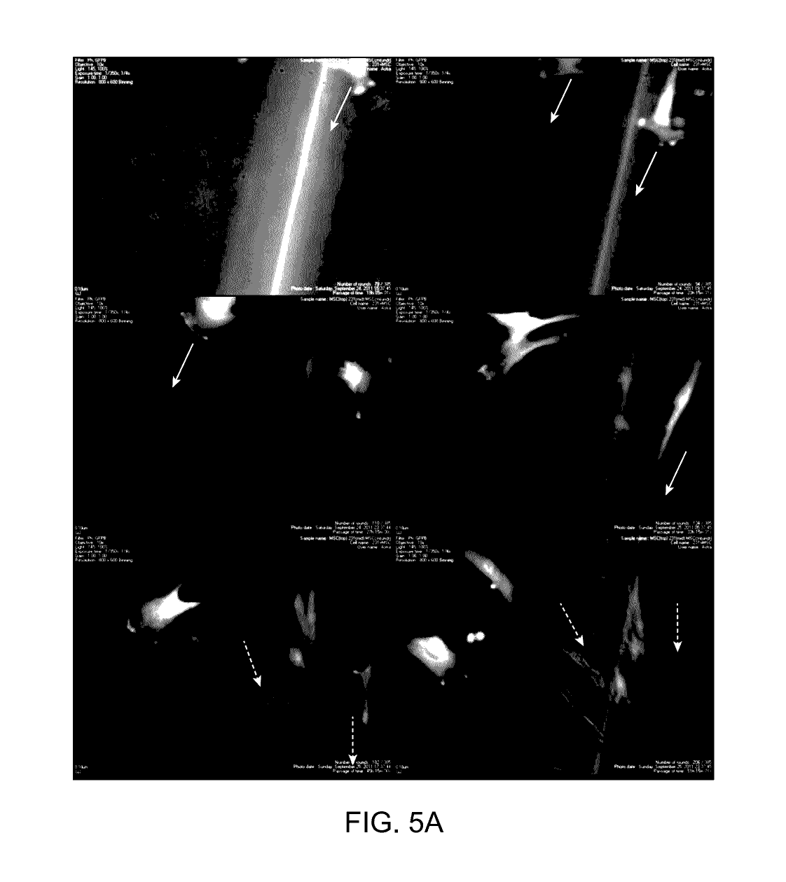

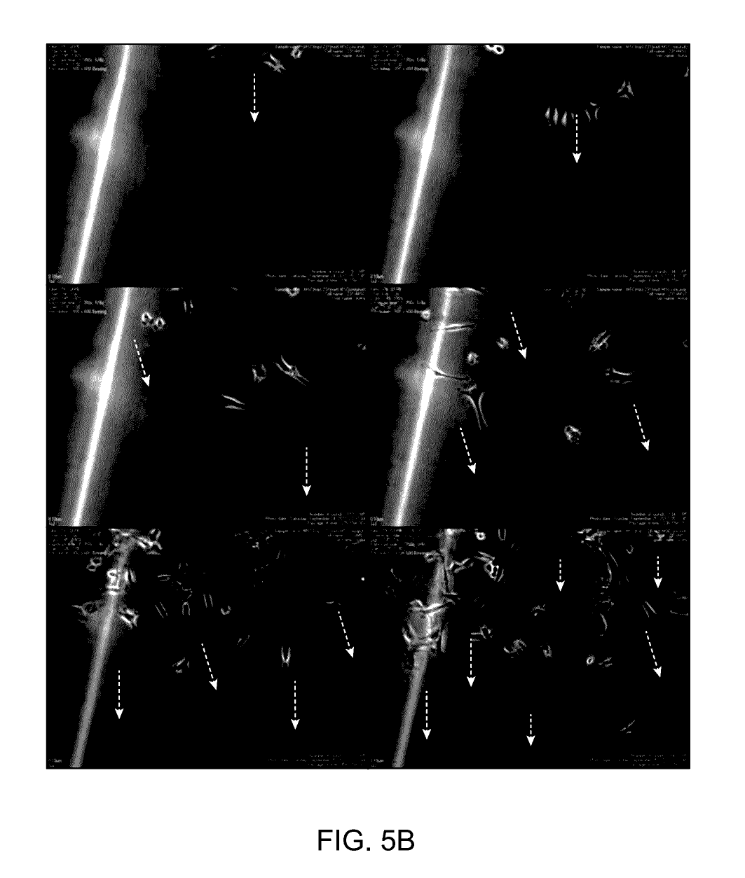

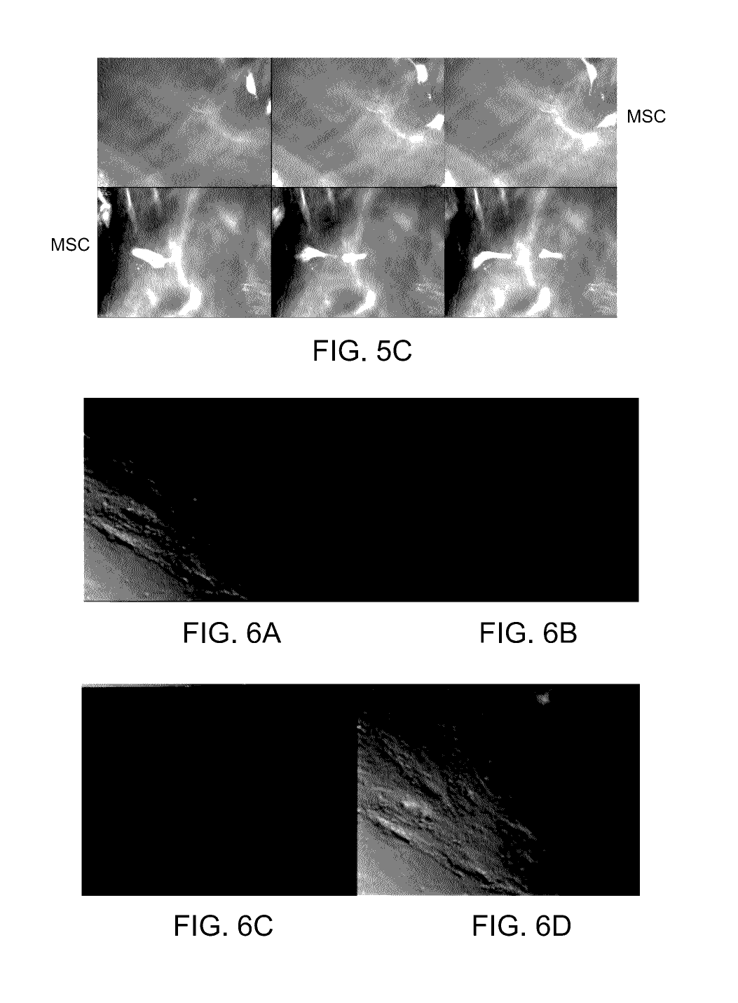

Migration of GFP⁺MSC toward cancer cells was assayed by under agar assay, and visualized by fluorescence and time-lapse video microscopy. GFP⁺MSC migrated toward MDA-MB-231 cancer cells (FIG. 5A) whereas GFP⁺MSC cultured in breast cancer conditioned media (GFP⁺MSCcm) attracted the MDA-MB-231 cells (FIG. 5B). GFP⁺MSC also preferentially migrated toward IL-6 over OPN (FIG. 5C).

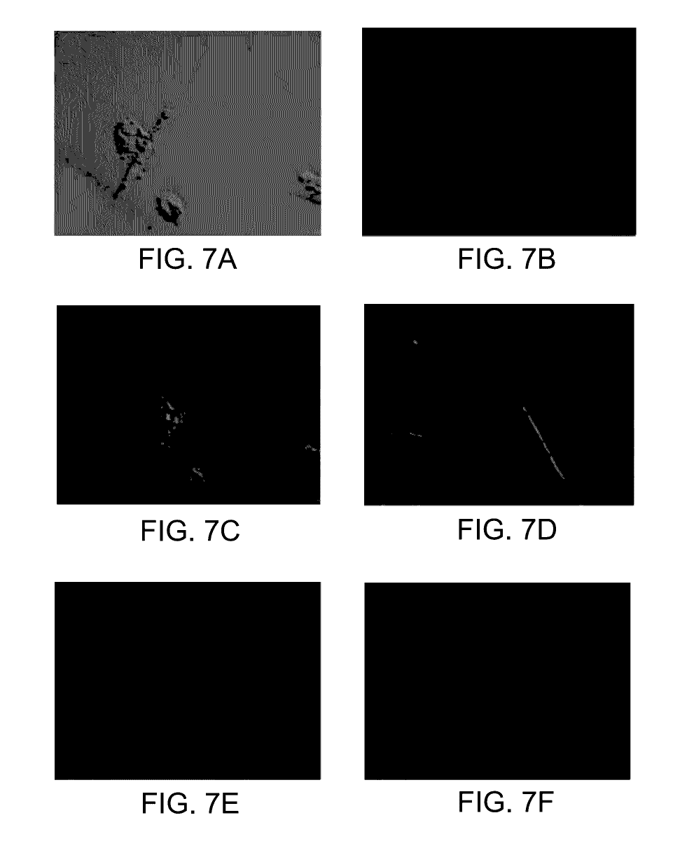

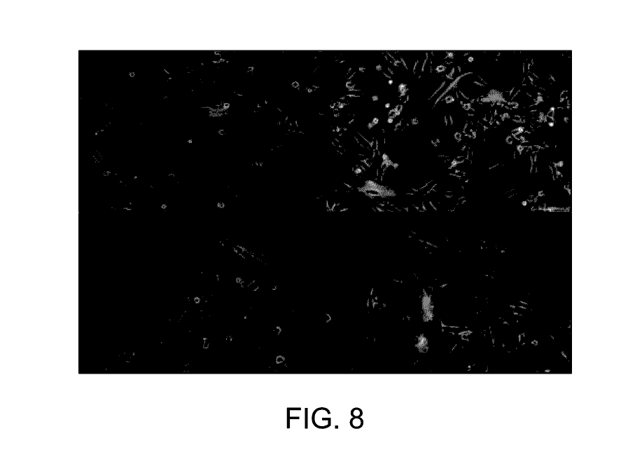

Following transduction, MSC transduced with the vector having the EF1α promoter displayed some expression of mCherry and CFP after approximately 5 days (FIG. 6). However, antibodies were indeed secreted into the culture media as demonstrated in that MDA-MB-231 cells incubated with supernatant from the engineered MSC and observed under fluorescence microscopy were labeled with the CFP, demonstrating that the antibody is bot secreted and will bind to the cancer cells (FIG. 7). In FIGS. 7 d, e, and f, CFP-tagging appears localized to a thin pseudopod-like projection from one of the cells, which is of particular interest since CD44v6 has been shown to be located at the invasive edge of metastatic cells. MSC transduced with the vector having the cancer specific CCL5 promoter and then co-cultured with MDA-MB-231 cells demonstrated stimulated expression in that mCherry was indeed observed (FIG. 8). These cells are candidates to be deemed ‘CD44v6 antibody secreting Mesenkillers’.

### Discussion

Monoclonal antibodies have been readily produced for a number of years and have significant research applications in a vast number of studies and assays. However clinical applications of therapeutic antibodies for the treatment of numerous diseases is hampered by a strong general immune response in patients. Through the selective targeting of a tumor-specific expressed protein (CD44v6), the stimulation of expression only within the tumor environment (through the use of the CCL5 promoter), and a carrier with intrinsic tumor-homing properties (MSC) Applicants have designed a biotherapeutic to overcome this obstacle. The therapeutically engineered CD44v6-antibody secreting MSC disclosed herein will tag metastasizing cancer stem cells while at the same time stimulating native immune attack.

Stimulated secretion of the antibody will occur under control of the CCL5/RANTES promoter. CCL5 is a secreted inflammatory chemokine that plays roles in immune function in part to stimulate migration of blood monocytes and CD4⁺/UCHL1⁺T lymphocytes (precursors of T-helper cells) involved in memory T-cell function (Schall, T. J. et al. (1990) Nature 347:669-671). Such processes occur in areas of injury to which MSC naturally home. Although physiologically, inflammatory cells and mediators (e.g., CCL2, CCL5) are immunological in nature, they have been shown to aid in cancer progression through their tumor promoting signaling and likely also through selective pressures leading to the survival and proliferation of tumor cells that have progressed in malignant transformation to have gained the ability to exploit immune function. The persistence of inflammatory mediators within the tumor environment supports tumor growth, proliferation, and motility (Ben-Baruch, A. (2011) Cancer Microenvironment). A key example of the tumor-exploitation of the immune system is given by the attraction of MSC to the tumor site and stimulated secretion of CCL5 from MSC. Just as CCL5 causes the mobilization of blood monocytes and T cells, MSC-secreted CCL5 will stimulate the migration of cancer cells (Karnoub, A. E. et al. (2007) Nature 449:557-563).

The precise mechanism through which cancer cells stimulate the secretion of CCL5 from the MSC is not fully understood however it is thought that tumor-derived osteopontin (OPN) is the stimulating signaling molecule whereby OPN is highly expressed in tumors cells and acts on MSC to cause the upregulation of CCL5 expression and secretion from MSC (Mi, Z. et al. (2011) Carcinogenesis 32:477-487). Here Applicants have confirmed upregulation of CCL5 expression and secretion by MSC in the presence of cancer cells (FIG. 4).

Although it is well documented that MSC will home to areas of hypoxia including the tumor environment, how cancer cells achieve this attraction is slowly coming to light. MSC trafficking toward regions of hypoxia is enhanced through chemoattractants such as IL-6, CCL2, PDGF and VEGF-A (which act synergistically), HGF, SDF-1, and IGF-1, which are released from areas of injury and inflammation, as well as tumor cells. Secretion of IL-6 (interleukin 6) from cancer cells is especially upregulated by hypoxia. IL-6 is an inflammatory cytokine that normally plays a role in the immune response and in inflammation, in part as a result of hypoxic conditions, and is thought to act in a paracrine fashion to recruit and activate MSC (Rattigan, Y. et al. (2010) Exp. Cell Res. 316:3417-3424). Applicants have shown here in vitro, that MSC will be preferentially attracted toward IL-6 over OPN. However, it is noted that the design of the experiment is in vitro, thus signaling is at relatively close distances (<1000 μm). In an in vivo setting IL-6 may act at greater distances in recruiting MSC to the general area, and other chemoattractants may also act within the tumor in calling the MSC toward the cancer cells.

After confirming dramatic increases in CCL5 production and secretion in MSC under the influence of cancer cells, Applicants have genetically engineered human bone marrow MSC to be “Mesenkillers” expressing a fusion protein made up of a single chain antibody against CD44v6 fused with Fc portion of the human IgG1 and a CFP tag. In the first vector Applicants used a ubiquitous EF1\acute{α} promoter to drive expression of the fusion protein antibody. The commonly utilized CMV promoter in lentiviral systems for ubiquitous expression of transduced genes of interest in various research applications, is often silenced in certain cell types such as hematopoietic and stem cells (\check{S}kalamera, D. et al. (2013) PLoS One 7:e51733), including MSC. Alternatively the EF1\acute{α} promoter has proven to allow for constitutive, long-term expression of genes of interest in these cells (\check{S}kalamera, D. et al. (2013) PLoS One 7:e51733; Serafini, M. et al. (2004) Haematologica 89:86-95). Thus this ubiquitous promoter allowed us to perform initial evaluations to determine whether the MSC will indeed express and secrete the engineered fusion antibody protein.

The constitutive EF1α promoter was replaced by the CCL5 promoter (approximately 800 bp in length). In doing so, Applicants were able to achieve selective induction of the CCL5 promoter and therefore antibody expression in the presence of cancer cells. When the signal arrived from the cancer cells to activate CCL5 expression, it also acted to drive the expression of the anti-CD44v6 engineered antibody. Fusion of the antibody with CFP facilitated the ability to follow the secreted antibodies and their tagging of the cancer cells. By including an IRES element Applicants aimed to simultaneously induce expression of a membrane targeted mCherry RFP, so that the membrane of the MSC would light up red as it is expressing the engineered antibodies, further facilitating the ability to evaluate the efficacy of the molecular engineering. Antibody tagging was evaluated by fluorescence microscopy and flow cytometry. Further characterization may be carried out using ELISA, or other analytic biochemical assay. Unfortunately, it appears that while the antibody was successfully detected, mCherry expression was weak at best, or not present, which may be due to shortcomings of the IRES element.

Sequences encoding the variable region of the antibody that allows for specific binding to CD44v6 were determined by proprietary methods by DNA2.0 from the amino acid sequence of a functional single chain antibody comprising only the variable heavy chain region (scFv). This single chain anti-human CD44v6 scFv was selected from a human phage-displayed scFv library based on its ability to bind in vitro to CD44v6 antigen analyzed by immunofluorescence, Western blot, and flow cytometry with an equilibrium dissociation constant (KD) determined to be 7.85±0.93×10⁻⁸M 2.

The anti-human CD44v6 scFv sequence was inserted in frame into a plasmid with sequences encoding the Fc and hinge regions of a human IgG molecule (InvivoGen). The Fc region was designed to stimulate increased antibody-dependent cytotoxicity (ADCC) and complement-dependent cytotoxicity (CDC). The signal sequence of Interleukin-2 (IL-2) was also provided (InvivoGen) and was included in the vector of the current study to direct secretion of the expressed molecule. IL-2 is a cytokine that signals in immune function to regulate leukocyte activity in response to microbial infections and foreign (non-self) elements (Smith, K. A. et al. (1980) Nature 287:853-855). The IL-2 signal sequence is made up of 21 amino acids that have common characteristics with similar signal sequences of other secreted proteins. This signal sequence is expressed along with the protein and is cleaved in the cytoplasm, leading to the secretion of the engineered protein.

CD44 is a transmembrane glycoprotein that interacts with the extracellular matrix at its amino terminal domain and cytoskeletal proteins at its carboxyl terminal domain thus mediating responses of cells to their microenvironment (Ponta, H. et al. (2003) Nature Reviews Molecular Cell Biology 4:33-45). The CD44 gene consists of 20 exons, the middle 10 of which are expressed via alternative splicing as variant forms. The standard form of CD44 (CD44s) lacks the variable exons and is widely expressed in several different cell types being distributed in wild type epithelial, mesenchymal and hematopoietic cells. CD44s is involved in the regulation of organ differentiation during development and in the maintenance of various tissues (Li, J. et al. (2012) Biomedicine and Pharmacotherapy 66:144-150; Borland, G. et al. (1988) Immunology 93:139-148; Sneath, R. J. et al. (1998) Journal of Clinical Pathology: Molecular Pathology 51:191-200; Goodison, S. et al. (1999) Journal of Clinical Pathology: Molecular Pathology 52:189-196). On the other hand, isoforms expressing the variable exons (variant forms) are primarily restricted to a certain tissue type (Mackay, C. R. et al. (1994) Journal of Cell Biology 124:71-82; Sherman, L. et al. (1998) Genes and Development 12:1058-1071). CD44 has also been shown to be involved in tumor necrosis factor (TNF) signaling. Interestingly TNF-α is abundant in the cancer microenvironment but has an inverse relationship with CD44s, whereas increases in TNF-α expression correlate with an upregulation of the variant form CD44v6. In particular, the variant 6 isoform (CD44v6) which expresses the variant exons 4 through 7, has been found predominantly located on various different types of carcinomas and has been shown to be involved in the metastasis of cancers (Ponta, H. et al. (2003) Nature Reviews Molecular Cell Biology 4:33-45; Günthert, U. (1991) Cell 65:13-24).

CD44v6 is localized on metastasizing cells on the invasive edge (invadopodia) (Xu, Y. P. et al. (2003) Journal of Zhejiang University Science 4:491-501) and has been shown to interact with the matrix metalloprotease MMP-9, a potent enzyme capable of breaking down extracellular matrix and remodeling tissue, lending further evidence that CD44v6 is actively enabling specific cancer cells to mobilize (Xu, Y. P. et al. (2003) Journal of Zhejiang University Science 4:491-501). On cancer cells, CD44v6 is also a co-receptor for the receptor of hepatocyte growth factor (HGF, also known as scatter factor [SF]) resulting in activation of the c-Met oncogenic signaling pathway with ligand binding, which indicates that CD44v6 contributes to the interference of apoptotic pathways and increased cancer cell survival. (Matzke, A. et al. (2007) Molecular and Cellular Biology 27:8797-8806; Toole, B. P. et al. (2008) Drug Resistance Updates 11:110-121; Comoglio, P. M. et al. (2008) Nature Reviews Drug Discovery 7:504-516) Furthermore CD44v6 regulates the actions of the miR-373 microRNA which has been shown to demonstrate pro-metastatic properties (Ma, L. et al. (2008) Trends in Genetics 24:448-456; Ventura, A. et al. (2009) Cell 136:586-591) and interestingly, aids in the assembly of a soluble matrix which allows exosomes from cancer cells to help prepare the pre-metastatic niche through modulation of (pre)metastatic organ cells for tumor cell embedding and growth (Jung, T. et al. (2009) Neoplasia 11:1093-1105). Adding to the complexity of CD44v6 in cancer progression and metastasis, the expression of CD44v6 is thought to be dynamic on cancer stem cells revealing itself on the cellular membrane at specific times of invasion and metastatic growth.

Increased HIF-1α expression and activation in cancer cells will result in increased expression of the receptor for CCL5, namely CCR5 (Lin, S. et al. (2012) Cancer Sci. 103:904-912). However, it is very interesting that secreted CCL5 will additionally interact with CD4442 (Roscic-Mrkic, B. et al. (2003) Blood 102:1169-1177). CCL5 binds with CD44 on the cancer cells and signals to enhance their mobility, invasive properties, and proliferation, resulting in an enrichment of tumor initiating cancer stem cells (Zhang, Y. et al. (2009) Oncol. Rep. 21:1113-1121) likely through a novel CD44-intracytoplasmic domain response element. MSC increase HIF1-α expression in response to the hypoxic environment. Of note, however, cancer cells do not require a hypoxic environment to activate expression of HIF-1α genes. Evidence has indicated that the CD44 intracytoplasmic domain (CD44-ICD) cleaves apart from the transmembrane protein, translocating itself within the nucleus where it is capable of activating HIF-1α responsive genes independent of a hypoxic environment, by binding to novel DNA consensus sequences that constitute a CD44-ICD response element in the promoter region of these genes. The expression of these genes results in an increase in cancer cell motility, increased cell survival, and tendency to undergo differentiation (Miletti-Gonzalez, K. E. et al. (2012) J. Biol. Chem. 287:18995-19007).

Specifically, the variant CD44v6 is expressed on cancer cells, most notably tumorigenic cancer stem cells. With its numerous roles in the progression and metastasis of cancer, Applicants submit that disrupting CD44v6 will hamper malignant progression. Moreover, since cancer stem cells are responsible for the seeding of metastatic tumors, the Mesenkiller cells disclosed herein will tag micrometastases, overcome their stealth and allow the innate immune function to clear them before being clinically relevant.

It should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A method for one or more of:
a. delivering a fusion polypeptide to a tumor cell expressing CD44 marker; and/or
b. for inducing an immune response in a subject in need thereof; and/or,
c. for inhibiting the growth of a tumor in a subject in need thereof;
comprising contacting the tumor cell or administering to the subject with an effective amount of an isolated host cell comprising an isolated polynucleotide, wherein the polynucleotide comprises a promoter element that drives expression of the C—C motif ligand 5 (“a CCL5 promoter”) operatively linked to a polynucleotide encoding the fusion polypeptide, the fusion polypeptide comprising the Fc region of a human antibody selected from the group: IgG1, IG2, IgG3, IgG4, IgA1, IgA2 or IgM and a ScFv region of an anti-CD44 polypeptide, or an equivalent of each thereof.

2. The method of claim 1, wherein the ScFv region of the CD44 is a ScFV region of CD44v6, that optionally has been codon-optimized.

3. The method of claim 1, wherein the isolated polynucleotide further comprises a polynucleotide encoding the signal sequence of a secreted protein.

4. The method of claim 3, wherein the signal sequence comprises an amino acid comprising the signal sequence of Interleukin-2 (IL-2), or an equivalent thereof.

5. The method of claim 1, wherein promoter element comprises from about nucleotides 2183 to about 3554 of SEQ ID NO. 1, or a fragment or an equivalent thereof that drives expression of a polynucleotide.

6. (canceled)

7. The method of claim 1, wherein the polynucleotide further comprises a polynucleotide encoding a membrane targeting signal sequence.

8. (canceled)

9. The method of claim 1, wherein the polynucleotide, further comprises a detectable label or a polynucleotide encoding a detectable label.

10-11. (canceled)

12. The method of claim 1, wherein the isolated polynucleotide further comprises an enhancer, optionally a WPRE polynucleotide.

13-15. (canceled)

16. The method of claim 1, wherein the polynucleotide further comprises a vector, that is optionally a plasmid or a viral vector.

17. An isolated polynucleotide, comprising, or alternatively consisting essentially of, or yet further consisting of, a promoter element that drives expression of the C—C motif ligand 5 (“a CCL5 promoter”) operatively linked to a polynucleotide encoding the fusion polypeptide, the fusion polypeptide comprising the Fc region of a human antibody selected from the group: IgG1, IG2, IgG3, IgG4, IgA1, IgA2 or IgM and a ScFv region of an anti-CD44 polypeptide, or an equivalent of each thereof.

18. The isolated polynucleotide of claim 17, wherein the ScFv region of the CD44 is a ScFV region of CD44v6, that optionally has been codon-optimized.

19. The isolated polynucleotide of claim 17, wherein the isolated polynucleotide further comprises a polynucleotide encoding the signal sequence of a secreted protein.

20. The isolated polynucleotide of claim 19, wherein the signal sequence comprises an amino acid comprising the signal sequence of Interleukin-2 (IL-2), or an equivalent thereof.

21. The polynucleotide of claim 17, wherein the promoter element comprises from about nucleotides 2183 to about 3554 of SEQ ID NO. 1, or a fragment or an equivalent thereof that drives expression of a polynucleotide.

22. (canceled)

23. The polynucleotide of claim 17, wherein the polynucleotide further comprises a polynucleotide encoding a membrane targeting signal sequence.

24. (canceled)

25. The polynucleotide of claim 17, further comprising a detectable label or a polynucleotide encoding a detectable label.

26-27. (canceled)

28. The polynucleotide of claim 17, further comprising an enhancer, optionally a WPRE polynucleotide.

29-31. (canceled)

32. The polynucleotide of claim 17, wherein the polynucleotide further comprises a vector, that is optionally a plasmid or a viral vector.

33. A isolated host cell comprising the isolated polynucleotide of claim 17.

34. An isolated polypeptide expressed from the isolated polynucleotide of claim 17.

35. (canceled)

36. The isolated host cell of claim 33, wherein the cell is an isolated stem cell, that is optionally a mesenchymal stem cell.

37-40. (canceled)

41. The isolated host cell of claim 36, characterized by the markers CD73, CD166, nucleostemin, CD44, CD45, CD90, CD45RO, CD105, CD54, CD49a, CD49e, CD51, CD29, CD56, Sca-1, SCF R/c-kit, SSEA-4, STRO-1, TfR, CD106, vimentin, and/or having the capacity to differentiate into an adipose cell, a bone cell, a cartilage cell, and a muscle cell or tissues comprising one or more of the cells.

42. An isolated population of substantially homogenous host cells of claim 33, that is optionally a clonal population.

43. A composition comprising: 1) a carrier and 2) one or more of: an isolated polynucleotide of claim 17.

44-52. (canceled)

53. The isolated polynucleotide of claim 17, wherein an equivalent thereof comprise a polynucleotide having at least 80% sequence identity or a polynucleotide that hybridizes under moderate or high stringency conditions, wherein moderate stringency comprises about 50° C. in about 6×SSC, and high stringency hybridization comprises at about 60° C. in about 1×SSC, to the reference polynucleotide or its complement.

54-56. (canceled)


SEQUENCE LISTING (13 sequences)

SEQ ID NO: 1 - DNA, 10838 bp, Artificial Sequence
  Description of Artificial Sequence Synthetic polynucleotide
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgccggtctgggccttggtttcctcttgtgcaactgaagaacatggaaaaaatgatccctaaagtcctttgaagctttcatattctgtaacttttgtgcccaagaaggccttacagtgagatgggatcccagtatttattgagtttcctcattcataaaatggggataataatagtaaatgagttgacacgcgctaagacagtggaacagtggctggcacagataagccctcggtaaatggtagccaataatgatagagtatgctgtaagatatctttctctccctctgcttctcaacaagtctctaatcaattattccactttataaacaaggaaatagaactcaaagacattaagcacttttcccaaaggtcgcttagcaagtaaatgggagagaccctatgaccaggatgaaagcaagaaattcccacaagaggactcattccaactcatatcttgtgaaaaggttcccaatgcccagctcagatcaactgcctcaatttacagtgtgagtgtgctcacctcctttggggactgtatatccagaggaccctcctcaataaaacactttataaataacatccttccatggatgagggaaaggaggtaagatctgtaatgaataagcaggaactttgaagactcagtgactcagtgagtaataaagactcagtgacttctgatcctgtcctaactgccactccttgttgtccccaagaaagcggcttcctgctctctgaggaggaccccttccctggaaggtaaaactaaggatgtcagcagagaaatttttccaccattggtgcttggtcaaagaggaaactgatgagctcactctagatgagagagcagtgagggagagacagagactcgaatttccggaggctatttcagttttcttttccgttttgtgcaatttcacttatgataccggccaatgcttggttgctattttggaaactccccttaggggatgcccctcaactggccctataaagggccagcctgagctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatgtaagtcctggtcttgaccaccacagcccctggagtcagactctgcctagaatgcctaccccatcccaatgactgtcccacaccttgccctcttttcaactgtagcatgaatactgtctgaaccccattatttacattcttatgaattgtcttacccatatgtcctgttgtccttgagagatctctatgctgcttcatggcagggatctcctgataccggggccgcctgagatcaccggcgaaggagggccaccatgtacaggatgcaactcctgtcttgcattgcactaagtcttgcacttgtcacgaattcggatatcgaggtccaactcgtacagtcaggagcagaagccaagaaacccggagcgtcagtgaaagtctcgtgcaaggcgtcgggttacacctttacgtcatatggaatctcgtgggtccgacaggccccagggcaggggcttgagtggatgggttggatttccgcgtataatggaaacaccaactacgcgcaaaagttgcagggaagagtgacaatgacgaccgatacttcgacaagcacagcctatatggaactgaggagccttcggtcggacgacacggcggtgtactactgtgcacgcggtccgtattccagctccttggattactgggggcagggcacactcgtgactgtatcgtcggggtccgcttcagcacccacgctgttccctctcgtcagatctatatcggccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctagggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatgctgtgctgtatgagaagaaccaaacaggttgaaaagaatgatgaggaccaaaagatcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 2 - DNA, 735 bp, Homo sapiens
gaggtacaactgcaggagtcaggacctggcctcgtgaaaccttctcagtctctgtctctcacctgctctgtcactggctactccatcaccagtggttattactggaactggatccggcaatttccaggaaacaaactggaatggatgggctacatgagaaacgacggtaataataactacaacccatctctcaaaaatcgactctccatcagtcgtgacacatcaaagaaccagtttttcctgaacttgaattctgtgactactgaggacacatctacatattactgtgcaagtcacggctacggtagtagcgggtttgtttactggggccaagggaccacggtcaccgtttcctctggcggtggcggttctggtggcggtggctccggcggtggcggttctgacatccagctgacccagtctgcactctccctgcctgtcagtcttggagatcaagcctccatctcttgcagatctagtcagagccttgtacacattaatggaaacacctatttacattggtacctgcagaagccaggccagtctccgaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgatcctccgacgttcggtggaggcaccaagctggagatc

SEQ ID NO: 3 - DNA, 696 bp, Homo sapiens
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 4 - DNA, 669 bp, Homo sapiens
acatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggccccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 5 - DNA, 2010 bp, Unknown
  Description of Unknown Wild-type IgG2 polynucleotide
agctttctggggcgagccgggcctgactttggctttggggcagggagtgggctaaggtgaggcaggtggcgccagccaggtgcacacccaatgcccgtgagcccagacactggaccctgcctggaccctcgtggatagacaagaaccgaggggcctctgcgccctgggcccagctctgtcccacaccgcggtcacatggcaccacctctcttgcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgacctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaaggtgggacccgcggggtatgagggccacatggacagaggccggctcggcccaccctctgccctgggagtgaccgctgtgccaacctctgtccctacagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggccagcaagcccccgctccccaggctctcggggtcgcgcgaggatgcttggcacgtaccccgtgtacatacttcccgggcacccagcatggaaataaagcacccagcgcttccctgggcccctgcgagactgtgatggttctttccacgggtcaggccgagtctgaggcctgagtggcatgagggaggcagagtgggtc

SEQ ID NO: 6 - DNA, 1944 bp, Artificial Sequence
  Description of Artificial Sequence Synthetic polynucleotide
ttcaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttggtgagaggccagcgcagggagggagggtgtctgctggaagccaggctcagccctcctgcctggacgcatcccggctgtgcagtcccagcccagggcaccaaggcaggccccgtctgactcctcacccggaggcctctgcccgccccactcatgctcagggagagggtcttctggctttttccaccaggctccgggcaggcacaggctggatgcccctaccccaggcccttcacacacaggggcaggtgctgcgctcagagctgccaagagccatatccaggaggaccctgcccctgacctaagcccaccccaaaggccaaactctctactcactcagctcagacaccttctctcttcccagatctgagtaactcccaatcttctctctgcagagctcaaaaccccacttggtgacacacctcccccatgcccacggtgcccaggtaagccagcccaggcctcgccctccagctcaaggcgggacaagagccctagagtggcctgagtccagggacaggccccagcagggtgctgacgcatccacctccatcccagatccccgtaactcccaatcttctctctgcagagcccaaatcttgtgacacacctcccccgtgcccaaggtgcccaggtaagccagcccaggcctcgccctccagctcaaggcaggacaggtgccctagagtggcctgcatccagggacaggtcccagtcgggtgctgacacatctgcctccatctcttcctcagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagctgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaagccaaaggtgggacccgcggggtatgagggccacgtggacagaggccagcttgacccaccctctgccctgggagtgaccgctgtgccaacctctgtccctacaggacagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgctacacgcagaagagcctctccctgtctccgggtaaatgagtgcggtacccgggaagccccgggtggcatccctgtgacccctccccagtgcctctcctggccctggaagttgccactccagtgcccaccagccttgtcctaataaaattaagttgcatcattttgtctgactaggtgtccttctataatatt

SEQ ID NO: 7 - DNA, 2033 bp, Artificial Sequence
  Description of Artificial Sequence Synthetic polynucleotide
gtacacccagaggtttaaggacaaggccaaactgactgcagtcacatccgccaacactgcctacatggagctcagcagcctgacaaatgaggactctgcggtctattactgttcaatcatctactttgattacgccgactttattatggactactggggccaagggaccacggtcaccgtctcgacaggtgagtgcggccgcgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagkcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgarcggcaaggagtacaagtgcaaggtctccarcaaaggcctcccgtcctccatcgagaaaaccatctccaamgccamaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcagktggcaggaggggaatgtcttctcatgctccgtgatgcatgaggctctgcacaaccactacacacagaagagcctctccctgtctctgggtaaatgagtgcggtacccgggtggcatccctgtgacccctccccagtgcctctcctggccctggaagttgccactccagtgcccaccagccttgtcctaataaaattaagttgcatcattttgtctgact

SEQ ID NO: 8 - DNA, 1732 bp, Artificial Sequence
  Description of Artificial Sequence Synthetic polynucleotide
gcatccccgaccagccccaaggtcttcccgctgagcctctgcagcacccagccagatgggaacgtggtcatcgcctgcctggtccagggcttcttcccccaggagccactcagtgtgacctggagcgaaagcggacagggcgtgaccgccagaaacttcccacccagccaggatgcctccggggacctgtacaccacgagcagccagctgaccctgccggccacacagtgcctagccggcaagtccgtgacatgccacgtgaagcactacacgaatcccagccaggatgtgactgtgccctgcccaggtcagagggcaggctggggagtggggcggggccaccccgtcgtgccctgacactgcgcctgcacccgtgttccccacagggagccgccccttcactcacaccagagtggaccgcgggccgagccccaggaggtggtggtggacaggccaggaggggcgaggcgggggcatggggaagcatgtgctgaccagctcaggccatctctccactccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgaccgtgacctctgtggctgctacagcgtgtccagtgtcctgtcgggctgtgccgagccatggaaccatgggaagaccttcacttgcactgctgcctaccccgagtccaagaccccgctaaccgccaccctctcaaaatccggtgggtccagaccctgctcggggccctgctcagtgctctggtttgcaaagcatattcctggcctgcctcctccctcccaatcctgggctccagtgctcatgccaagtacasagggaaactgaggcaggctgaggggccaggacacagcccggggtgcccaccagagcagaggggctctctcatcccctgcccagccccctgacctggctctctaccctccaggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactgaggtacctgatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgc

SEQ ID NO: 9 - DNA, 1799 bp, Artificial Sequence
  Description of Artificial Sequence Synthetic polynucleotide
cctctctgtgctgggttcctccagtgtagaggagaggcaggtacagcctgtcctcctggggacatggcatgagggccgcgtcctcacagcgcattctgtgttccagcatccccgaccagccccaaggtcttcccgctgagcctcgacagcaccccccaagatgggaacgtggtcgtcgcatgcctggtccagggcttcttcccccaggagccactcagtgtgacctggagcgaaagcggacagaacgtgaccgccagaaacttcccacctagccaggatgcctccggggacctgtacaccacgagcagccagctgaccctgccggccacacagtgcccagacggcaagtccgtgacatgccacgtgaagcactacacgaattccagccaggatgtgactgtgccctgccgaggtcagagggcaggctggggagtggggcggggccaccccgtcctgccctgacactgcgcctgcacccgtgttccccacagggagccgccccttcactcacaccagagtggaccgcgggccgagccccaggaggtggtggtggacaggccaggaggggcgaggcgggggcacggggaagggcgttctgaccagctcaggccatctctccactccagttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggctgtgcccagccatggaaccatggggagaccttcacctgcactgctgcccaccccgagttgaagaccccactaaccgccaacatcacaaaatccggtgggtccagaccctgctcggggccctgctcagtgctctggtttgcaaagcatattcccggcctgcctcctccctcccaatcctgggctccagtgctcatgccaagtacagagggaaactgaggcaggctgaggggccaggacacagcccagggtgcccaccagagcagaggggctctctcatcccctgcccagccccctgacctggctctctaccctccaggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactgagccgcccgcctgtccccacccctgaataaactccatgctcccccaagcagccccacgcttccatccggcgcctgtctgtccatcctcagggtctcagcacttgggaaagggccagggcatggacagggaagaataccccctgccctgagcctcggggggcccctggcacccccatgagactttccaccctggtgtgagtgtgagttgtgagtgtgagagtgtgtggtgcaggagg

SEQ ID NO: 10 - DNA, 1453 bp, Artificial Sequence
  Description of Artificial Sequence Synthetic polynucleotide
gggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgccgagctgcctcccaaagtgagcgtcttcgtcccaccccgcgacggcttcttcggcaacccccgcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccggtaaacccaccctgtacaacgtgtccctggtcatgtccgacacagctggcacctgctactgaccctgctggcctgcccacaggctcggggcggctggccgctctgtgtgtgcatgcaaactaacccgtgtcaacggggtgagatgttgcatct

SEQ ID NO: 11 - DNA, 20 bp, Artificial Sequence
  Description of Artificial Sequence Synthetic primer
tgcagaggatcaagacagca

SEQ ID NO: 12 - DNA, 20 bp, Artificial Sequence
  Description of Artificial Sequence Synthetic primer
gagcacttgccactggtgta

SEQ ID NO: 13 - DNA, 594 bp, Unknown
  Description of Unknown WPRE polynucleotide
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatc